(19) United States
(12) Patent Application Publication
(10) Pub. No.: US 2004/0047868 A1
(43) Pub. Date: Mar. 11, 2004

(54) Treating endotoxemia and related disorders with probiotics

(76) Inventors: Gerald Pang, New SouthWales (AU); John Cade, Vic (AU); Robert Llewellyn Clancy, New South Wales (AU); Robert Batey, New South Wales (AU); Margaret Lorraine Dunkley, New South Wales (AU); Patricia Lynne Conway, New South Wales (AU)

Correspondence Address: FISH & RICHARDSON PC, 225 FRANKLIN ST, BOSTON, MA, 02110, US

(21) Appl. No.: 10/332,173

(22) PCT Filed: Jul. 3, 2001
(86) PCT No.: PCT/AU01/00796

(30) Foreign Application Priority Data
Jul. 3, 2000 (AU) PQ 8542
Jul. 6, 2000 (AU) PQ 8598

(51) Int. Cl. A61K 39/00; A61K 39/38

(52) U.S. Cl. 424/184.1

(57) ABSTRACT

The invention relates to methods of preventing and treating endotoxemia and related disorders. In particular, the invention relates to methods of preventing and treating endotoxemia and related disorders using a probiotic. The invention also relates to methods for assessing the efficacy ot a probiotic in the prevention and treatment of endotoxemia and related disorders.

### TECHNICAL FIELD

The invention relates to methods of preventing and treating endotoxemia and related disorders. In particular, the invention relates to methods of preventing and treating endotoxemia and related disorders using a probiotic. The invention also relates to methods for assessing the efficacy of a probiotic in the prevention and treatment of endotoxemia and related disorders.

### BACKGROUND

Any discussion of the prior art throughout the specification should in no way be considered as an admission that such prior art is widely known or forms part of common general knowledge in the field.

Endotoxins are lipopolysaccharides which are large (MW 200,000 to 1,000,000), heat stable molecules found in the cell walls of gram-negative bacteria. Colonisation of the gut by gram-negative bacteria—particularly E. coli but also other species—contributes small amounts of endotoxin into the circulation. A variety of pathological conditions that alter colonisation characteristics or mucosal integrity can markedly enhance bacterial translocation leading to high levels of circulating endotoxin i.e. endotoxemia.

Endotoxemia in turn is linked to a variety of clinical disease states due to tissue damage caused by endotoxin. Examples include acute disorders such as “endotoxic shock”, through to more subtle chronic tissue damage such as liver cell necrosis/(inflammation) (hepatitis), particularly under circumstances of co-administration of agents known to be linked to liver disease (eg. alcohol and hepatotoxic virus).

Recently, a common pathway towards liver cell damage has focussed on circulating T lymphocytes. Classically accepted as vehicles of hepatocyte damage are Hepatitis A, B and C infection as well as autoimmune hepatitis (and presumably other viral infections, eg. Ebstein Barr Virus (EBV), cytomegalovirus (CMV)). It now appears that T-lymphocytes are also involved in alcohol-related chronic liver disease. It has been found that alcohol enhances the secretion of tumour necrosis factor (TNF) from T lymphocytes. In the presence of a T-cell stimulant (notably endotoxin) this “double-hit” model ensures high concentrations of TNF within the liver, leading to cell death, hepatitis and, if the framework of the liver is disturbed, cirrhosis.

The contribution of E. coli to raised levels of endotoxin in alcoholic hepatitis has been demonstrated, with a fall in endotoxin levels in patients given antibiotics. Although antibiotics (oral) reduce levels of endotoxin, they are not an ideal way of achieving this end, particularly on a long-term basis. Other strategies considered for therapy include 3-deoxy-D-manno-2-octulosonate synthesis inhibitors, polymyxin B (a polycationic molecule which binds to endotoxin), plasmapheresis, monoclonal and polyclonal immunotherapy and treatment with antagonists of endotoxin (reviewed by J Hurley (1995) in Clin Microbiol Rev 8:268-292). Advantages and disadvantages are associated with each of these strategies but, as yet, no simple and effective therapy exists for endotoxemia.

It is an object of the present invention to overcome or ameliorate at least one of the disadvantages of the prior art, or to provide a useful alternative.

### SUMMARY OF THE INVENTION

It has surprisingly been found that treatment with a probiotic can reduce endotoxin levels and reduces alcohol-induced hepatic damage. The probiotic may be any one or more of a number of micoorganisms. Treatment with lactic acid bacteria is described below. Mammalian models that may be used to determine the efficacy of probiotics are also described.

According to a first aspect, the invention provides a A method for the prevention and/or treatment of endotoxemia including administering to a mammal in need of such treatment an effective amount of a probiotic, wherein the probiotic is probiotic bacteria selected from Lactobacillus species, with the proviso that the Lactobacillus species is not Lactobacillus GG.

The mammal may be at risk of developing endotoxemia or may already have developed endotoxemia. The mammal may be suffering from an acute condition, for example, a critical illness or acute hepatitis, or may be suffering from a chronic condition, for example, chronic liver disease.

It will be clear to the skilled addressee that the method and/or use may apply to any condition in which endotoxins are present in the circulation.

According to a second aspect, the present invention provides a method of treating an alcohol-induced liver disorder including administering a therapeutically effective amount of a probiotic wherein the probiotic is probiotic bacteria selected from Lactobacillus species, with the proviso that the Lactobacillus species is not Lactobacillus GG, to a mammal in need thereof.

According to a third aspect, the present invention provides a method of treating an inflammatory bowel disease including administering a therapeutically effective amount of a probiotic to a mammal in need thereof.

According to a fourth aspect, the present invention provides use of a method for the prevention and/or treatment of endotoxemia associated with inflammatory bowel disease including administering to a mammal in need of such treatment an effective amount of a probiotic.

According to a fifth aspect, the present invention provides use of a probiotic for the manufacture of a medicament for the prevention and/or treatment of endotoxemia, wherein the probiotic is probiotic bacteria selected from Lactobacillus species, with the proviso that the Lactobacillus species is not Lactobacillus GG.

According to a sixth aspect, the present invention provides use of a probiotic in the preparation of a medicament for the prevention and/or treatment of an alcohol-induced liver disorder, wherein the probiotic is probiotic bacteria selected from Lactobacillus species, with the proviso that the Lactobacillus species is not Lactobacillus GG.

According to a seventh aspect, the present invention provides use of a probiotic in the preparation of a medicament for the prevention and/or treatment of inflammatory bowel disease.

According to an eighth aspect, the present invention provides use of a probiotic in the preparation of a medicament for the prevention and/or treatment of endotoxemia associated with inflammatory bowel disease.

The mammal is preferably a human, but it will be clear to the skilled addressee that other mammals are also contemplated.

Suitable probiotics may be determined by the skilled addressee and include, for example, lactic acid bacteria such as Lactobacillus. Preferably, the probiotic is Lactobacillus acidophilus or Lactobacillus fermentum. Most preferably, the probiotic is L. acidophilus strain (VRI-001) or L. fermentum strain (VRI-002). Both strains are obtainable from University of New South Wales, School of Microbiology and Immunology Culture Collection, Sydney, Australia.

Preferably, the probiotic or medicament is administered orally although other routes of administration are also contemplated, eg. by suppositories, or through the gastric route by intubation.

Preferably, the probiotic or medicament is in the form of a tablet or capsule although it may also be in the form of a food composition, eg. a dairy or soy product. Preferably, the tablet or capsule contains 10⁸ to 10¹² cfu of probiotic.

According to a ninth aspect, the present invention provides a method for assessing the effect of a probiotic in the prevention and/or treatment of endotoxemia in a mammal including:

(a) administration of an endotoxin and/or an endotoxin-inducing agent to, and/or inflicting an endotoxin-inducing insult on, the mammal;

(b) administration of the probiotic to the mammal; and

(c) determination of the effect of the probiotic on endotoxemia, wherein the probiotic is probiotic bacteria selected from Lactobacillus species, with the proviso that the Lactobacillus species is not Lactobacillus GG.

According to a tenth aspect, the present invention provides a method for assessing the effect of a probiotic in the prevention and/or treatment of an alcohol-induced liver disorder in a mammal including:

(a) administration of alcohol to the mammal;

(b) administration of the probiotic to the mammal; and

(c) determination of the effect of the probiotic on the condition of the liver of the mammal, wherein the probiotic is probiotic bacteria selected from Lactobacillus species, with the proviso that the Lactobacillus species is not Lactobacillus GG.

According to an eleventh aspect, the present invention provides a method for assessing the effect of a probiotic in the prevention and/or treatment of inflammatory bowel disorder in a mammal including:

(a) administration of an agent that causes inflammation of the bowel;

(b) administration of the probiotic to the mammal; and

(c) determination of the effect of the probiotic on the condition of the bowel.

According to a twelfth aspect, the present invention provides a method of controlling circulating endotoxin levels in a mammal including inhibiting growth and/or translocation of enteric Gram-negative bacteria by administering a probiotic, wherein the probiotic is probiotic bacteria selected from Lactobacillus species, with the proviso that the Lactobacillus species is not Lactobacillus GG.

It will be clear to the skilled addressee that steps (a) and (b) of the method may be carried out simultaneously or sequentially and, when carried out sequentially the nature of the effect to be assessed will determine whether step (a) should be carried out before or after step (b).

The mammal may be any mammal including a human. The mammal may also be, for example, a laboratory animal model such as a rat.

The concentration, amount and means of delivery of the alcohol will depend on a variety of factors such as the effect being monitored, the type of mammal, the type and formulation of the probiotic, etc. The skilled addressee will be able to easily determine such parameters. For example, the alcohol may be administered to the mammal in increasing concentrations over a suitable period. Amounts of alcohol could, for example, range from, say, 5% to 40% and may be administered in drinking water. For example, the alcohol may be administered to the mammal over a period of months—for example 2 months.

Alternatively, for example, alcohol may b delivered to the colon of a mammal in order to induce inflammatory bowel disease. The appropriate concentration and amount of alcohol can be readily determined by the skilled addressee and may be, for example, 45% ethanol.

Preferably, the probiotic assessed is a lactic acid bacteria such as Lactobacillus. More preferably, the probiotic is Lactobacillus acidophilus or Lactobacillus fermentum and, most preferably, it is L. acidophilus strain VRI-001 or L. fermentum strain VRI-002. Both strains are obtainable from University of New South Wales, School of Microbiology and Immunology Culture Collection, Sydney, Australia.

Preferably, the probiotic is administered orally although other routes of administration are also contemplated, eg by suppositories or through the gastric route by intubation. Preferably, the probiotic is in the form of a tablet or capsule or sachet although it may also be in the form of a food composition, eg. a dairy or soy product. Preferably, the tablet or capsule contains 10⁸ to 10¹² cfu of probiotic.

When the mammal is a rat, the probiotic may be administered in an amount of about 5×10¹⁰ cfu every second day. Preferably, the probiotic is present in phosphate buffered saline (PBS). The probiotic may be administered, for example, by intragastric feed.

The effect of the probiotic may be determined by any suitable method. One such method is measurement of a parameter that is a direct and/or indirect indicator of the level of circulating endotoxin. The effect of the probiotic may, for example, be determined by measurement of endotoxin levels by a Limulus Amebocyte Lysate (LAL) assay.

The effect of the probiotic may, for example, be determined by measurement of a parameter indicative of the level of damage such as measurement of plasma alanine aminotransferase (ALT) which correlates positively with liver damage.

Preferably the endotoxin-inducing agent is alcohol. Preferably, the alcohol induces a liver disorder.

In the context of the present invention, the term “prevention and/or treatment of endotoxemia” includes, but is not limited to, prevention and/or treatment of increased endotoxin levels and/or conditions/complications caused by endotoxemia.

### BRIEF DESCRIPTION OF THE FIGURES

FIG. 1 Levels of alanine aminotransferase (ALT) in 8 rats fed with ethanol and phosphate buffered saline (PBS) containing L. acidophilus; and 8 rats fed with ethanol and PBS alone. The difference in ALT levels in the two groups was significant at the level of p<0.033.

FIG. 2 Levels of blood endotoxin in rats fed alcohol over a 28 day period measured by the Limulus Amebocyte Lystate (LAL) assay.

FIG. 3 Control of blood endotoxin levels by probiotic bacteria in mice with ethanol-induced inflammation of the colon.

FIG. 4 Reduction in enteric bacteria colonisation in the colon correlates with an absence of blood endotoxin in probiotic-fed mice following challenge with ethanol.

### DESCRIPTION OF THE INVENTION

Preferred embodiments of the invention are described below, by way of example only, with reference to the accompanying Figures.

### EXAMPLE 1

A rat model of alcohol-induced hepatic damage has previously been described (Cao Qi, Batey R, Pang G, Clancy R (1998) Alcoholism: Clinical and Experimental Research 22: 723-729). Using this model, it has surprisingly been found that administration of a probiotic can reduce liver damage.

Female Wistar rats (n=10-14), 8-10 weeks old and weighing approximately 250 g, were housed in individual hanging wire cages in a controlled temperature environment with a 12 hr light-dark cycle and were allowed free access to rat chow and drink throughout the study. The study was performed in accordance with regulations set down by the animal research ethics committee of the University of Newcastle, Australia.

The rats were fed ethanol at increasing concentrations in distilled water over a 4-week period, starting at 5% ethanol (v/v). When the concentration of ethanol in the distilled water reached 40% (v/v), this level was maintained for 4 further weeks. During the latter 4 weeks, each rat was also fed orally with L. acidophilus (5×10¹⁰ cfu per rat every second day) in phosphate buffered saline (PBS: 0.01 M phosphate buffer, 0.020 M potassium chloride and 0.120 M sodium chloride, at pH 7.4) delivered by intragastric feed. The L. acidophilus strain used VRI-001 which is obtainable from University of New South Wales, School of Microbiology and Immunology Culture Collection, Sydney, Australia.

Control rats (n=8) were treated in the same manner as described above with the exception that PBS alone (without Lactobacillus) was provided.

Blood plasma alanine aminotransferase (ALT) levels were determined (n=8) at the end of the 8-week period by a standard photometric method using a bichromatic analyzer (Department of Biochemistry, John Hunter Hospital, Newcastle, Australia). Plasma ALT levels are positively correlated with liver damage and measurement of the level of this enzyme in plasma is an accepted standard methodology by which to assess hepatic damage.

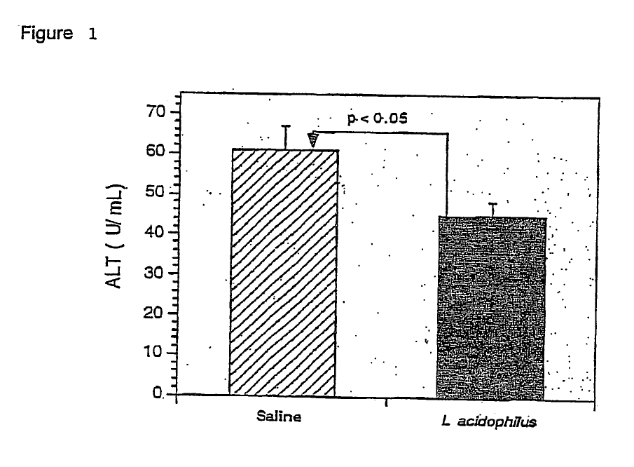

Results are presented in FIG. 1 and show that rats fed L. acidophilus had significantly less ALT than those not receiving the probiotic (p<0.03). Thus, administration of L. acidophilus can result in a reduction in liver damage. The alcohol-induced liver damage model may be used as a general liver damage model and, in particular, bears commonality not only in terms of hepatic lesion but also in terms of cellular response with disorders such as viral hepatitis. Results obtained can therefore be used as an indication of the effect of administration of a probiotic to those suffering from such disorders.

### EXAMPLE 2

The alcohol-induced hepatic damage rat model can also be used as a general model for endotoxemia since high levels of endotoxin are present in the circulation of these animals.

Lactic acid bacteria are gram-positive bacteria and reside transiently within the gastrointestinal tract. It has surprisingly been found that administration of supplementary lactic acid bacteria in the form of L. acidophilus reduces the level of circulating endotoxins in this animal model.

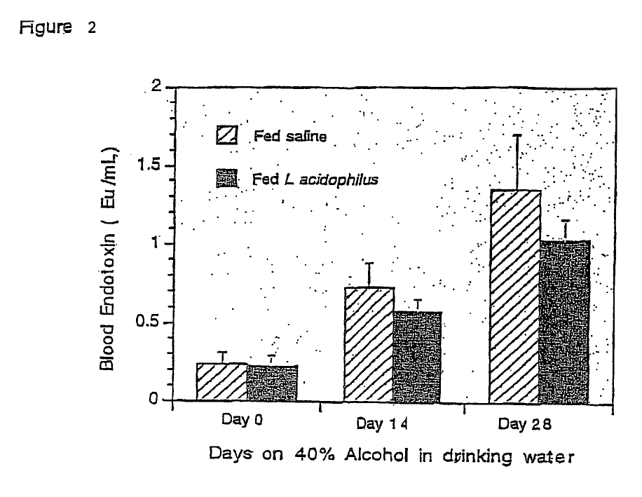

Blood samples were taken at 0, 14 and 28 days (after the first day on which 40% alcohol was administered) from the rats used in Example 1 by tail bleeding. Plasma endotoxin levels were measured using the Limulus Amebocyte Lysate (LAL) assay (discussed in J. C. Hurley (1995) Endotoxemia: Methods of Detection and Clinical Correlates, Clin Microbiol Rev 8:268-292) supplied by Cape Cod Inc, Woods Hole, Mass., USA. As shown in FIG. 2, lower levels of endotoxin were detected in rats fed L. acidophilus at day 14 and 28 compared to rats fed saline solution.

Without being bound by theory, it is therefore assumed that L. acidophilus is capable of regulating the translocation of endotoxin.

### EXAMPLE 3

To demonstrate that the ability to lower endotoxin levels was not confined to the rat alcohol-induced hepatic damage model described above, a mouse model of ethanol-induced inflammatory bowel disease was pre-treated with the probiotic L. fermentum to determine its effect on endotoxins induced by this condition.

Effect of Probiotic on Circulating Endotoxin

Female BALB/c mice (groups of 5, 8 weeks old) were fed 1×10⁹ Lactobacillus fermentum (strain VRI-002 obtainable from University of New South Wales, School of Microbiology and Immunology Culture Collection, Sydney, Australia) on three consecutive days before challenge with 45% ethanol solution administered via the rectum into the colon using a fine canulla. Control mice were fed PBS.

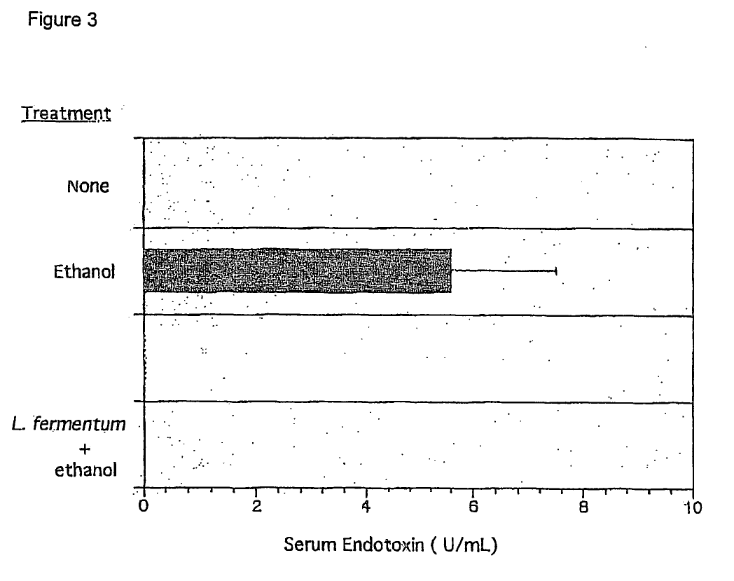

Two days after challenge, the mice were killed and blood plasma endotoxin levels analysed using the LAL assay described above. As shown in FIG. 3, mice receiving no treatment (normal) or mice fed L. fermentum before administration of ethanol had no detectable endotoxin in the blood whereas mice fed PBS and treated with ethanol had significantly higher levels of endotoxin.

The results indicate that in this mouse model of ethanol-induced inflammatory bowel disease administration of probiotics prior to inducing inflammatory bowel disease prevented the rise in endotoxin levels that normally accompanies this disease.

### EXAMPLE 4

Relationship Between Gut Flora Balance and Endotoxemia

To examine whether an increase or decrease in blood endotoxin correlates with the amount of gut flora present, the levels of colonisation of enteric and coliform bacteria in the faeces were determined. The faecal content of mice used in Example 3 were suspended in PBS and plated in serial 10-fold dilutions onto McConkey CM7 agar medium. After 2-3 days, colony counts were performed and the results were expressed as log₁₀ colony-forming units (CFU) per gm faeces.

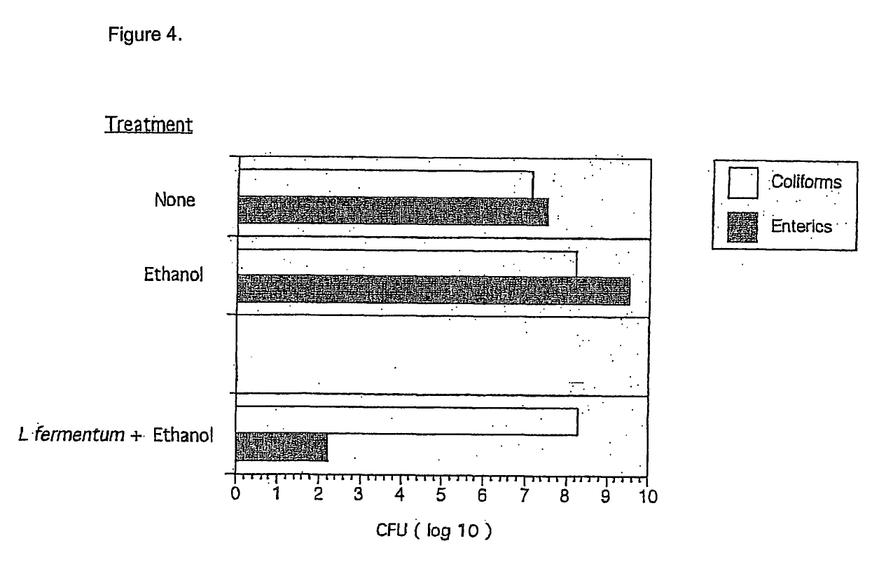

As shown in FIG. 4, while there was an increase in coliform (1 log) bacteria in the L. fermentum-fed group, a 6-7 log reduction in enteric forms in the colon was observed which correlated with no detectable endotoxin in the blood.

Without wishing to be bound by theory, these data show that probiotics are capable of controlling endotoxin levels in blood through inhibition of enteric gram-negative bacterial growth in the gut following stress.

Although the invention has been described with reference to specific examples, it will be appreciated by those skilled in the art that the invention may be embodied in many other forms.

The claims defining the invention are as follows:

1. A method for the prevention and/or treatment of endotoxemia including administering to a mammal in need of such treatment an effective amount of a probiotic, wherein the probiotic is probiotic bacteria selected from Lactobacillus species, with the proviso that the Lactobacillus species is not Lactobacillus GG.

2. A method according to claim 1 wherein the mammal is suffering from a hepatic disorder.

3. A method according to claim 2 wherein the hepatic disorder is hepatitis.

4. A method according to claim 3 wherein the hepatic disorder is alcohol-induced liver disease.

5. A method of treating an alcohol-induced liver disorder including administering a therapeutically effective amount of a probiotic wherein the probiotic is probiotic bacteria selected from Lactobacillus species, with the proviso that the Lactobacillus species is not Lactobacillus GG, to a mammal in need thereof.

6. A method of treating inflammatory bowel disease including administering a therapeutically effective amount of a probiotic to a mammal in need thereof.

7. A method for the prevention and/or treatment of endotoxemia associated with inflammatory bowel disease including administering to a mammal in need of such treatment an effective amount of a probiotic.

8. A method according to any one of claims 1 to 7 wherein the mammal is a human, horse, pig, a member of ovine, bovine or poultry species, or a companion animal such as a dog or a cat.

9. A method according to claim 6 or 7 wherein the probiotic is Lactobacillus.

10. A method according to any one of claims 1 to 5 or 9 wherein the Lactobacillus is L. acidophilus.

11. A method according to claim 10 wherein the L. acidophilus is strain VRI-001.

12. A method according to any one of claims 1 to 5 or 9 wherein the Lactobacillus is L. fermentum.

13. A method according to claim 12, wherein the L. fermentum is strain VRI-002.

14. A method according to any one of claims 1 to 13, wherein the probiotic is administered orally, by suppositories or through the gastric route by intubation.

15. A method according to claim 14 wherein the probiotic is in the form of a tablet, capsule, powder or liquid.

16. A method according to 15 wherein the tablet or capsule contains 10⁸ to 10¹² cfu of probiotic bacteria.

17. A method according to claim 14 wherein the probiotic is in the form of a food composition.

18. A method according to 17 wherein the food is a dairy or soy product.

19. A method according to claim 17 or claim 18, wherein the food composition contains 10⁸ to 10¹² cfu of probiotic bacteria per serve.

20. Use of a probiotic for the manufacture of a medicament for the prevention and/or treatment of endotoxemia, wherein the probiotic is probiotic bacteria selected from Lactobacillus species, with the proviso that the Lactobacillus species is not Lactobacillus GG.

21. Use of a probiotic in the preparation of a medicament for the prevention and/or treatment of an alcohol-induced liver disorder, wherein the probiotic is probiotic bacteria selected from Lactobacillus species, with the proviso that the Lactobacillus species is not Lactobacillus GG.

22. Use of a probiotic in the preparation of a medicament for the prevention and/or treatment of inflammatory bowel disease.

23. Use of a probiotic in the preparation of a medicament for the prevention and/or treatment of endotoxemia associated with inflammatory bowel disease.

24. Use according to claim 22 or claim 23, wherein the probiotic is a Lactobacillus.

25. Use according to claim 24 wherein the Lactobacillus is L. acidophilus.

26. Use according to claim 25 wherein the L. acidophilus is the strain VRI-001.

27. Use according to claim 24 wherein the Lactobacillus is L. fermentum.

28. Use according to claim 26 wherein the L. fermentum is strain VRI-002.

29. Use according to any one of claims 20 to 28, wherein the probiotic is for oral or rectal administration, or through the gastric route by intubation.

30. Use according to claim 29 wherein the probiotic is in the form of a tablet, capsule, powder or liquid.

31. Use according to claim 30 wherein the tablet or capsule contains 10⁸ to 10¹² cfu of probiotic bacteria.

32. Use according to claim 29 wherein the probiotic is in the form of a food composition.

33. Use according to 32 wherein the food is a dairy or soy product.

34. Use according to claim 32 or claim 33, wherein the food composition contains 10⁸ to 10¹² cfu of probiotic bacteria per serve.

35. A method for assessing the effect of a probiotic in the prevention and/or treatment of endotoxemia in a mammal including:
(a) administration of an endotoxin and/or an endotoxin-inducing agent to, and/or inflicting an endotoxin-inducing insult on, the mammal;
(b) administration of the probiotic to the mammal; and
(c) determination of the effect of the probiotic on endotoxemia, wherein the probiotic is probiotic bacteria selected from Lactobacillus species, with the proviso that the Lactobacillus species is not Lactobacillus GG.

36. A method for assessing the effect of a probiotic in the prevention and/or treatment of an alcohol-induced liver disorder in a mammal including:
(a) administration of alcohol to the mammal;
(b) administration of the probiotic to the mammal; and
(c) determination of the effect of the probiotic on the condition of the liver of the mammal, wherein the probiotic is probiotic bacteria selected from Lactobacillus species, with the proviso that the Lactobacillus species is not Lactobacillus GG.

37. A method for assessing the effect of a probiotic in the prevention and/or treatment of inflammatory bowel disorder in a mammal including:
(a) administration of an agent that causes inflammation of the bowel;
(b) administration of the probiotic to the mammal; and
(c) determination of the effect of the probiotic on the condition of the bowel.

38. A method according to any one of claims 35 to 37 wherein steps (a) and (b) are carried out simultaneously.

39. A method according to any one of claims 35 to 37 wherein steps (a) and (b) are carried out sequentially wherein step (a) may be carried out before or after step (b).

40. A method according to any one of claims 35 to 39 wherein the alcohol is administered to the mammal in increasing concentrations over a suitable period.

41. A method according to claim 40 wherein the amounts of alcohol range from 5% to 40% in drinking water.

42. A method according to claim 41 wherein the alcohol is administered to the mammal over a period of about two months.

43. A method according to any one of claims 35 to 42 wherein the alcohol is delivered to the colon of the mammal.

44. A method according to claim 43 wherein the alcohol is at a concentration of 45%.

45. A method according to any one of claims 37 to 44, wherein the probiotic is Lactobacillus.

46. A method according to any one of claims 35, 36 or 45 wherein the Lactobacillus is L. acidophilus.

47. A method according to claim 46 wherein the L. acidophilus is the strain VRI-001.

48. A method according to any one of claims 35, 36 or 45 wherein the probiotic is L. fermentum.

49. A method according to claim 48 wherein the L. fermentum is strain VRI-002.

50. A method according to any one of claims 35 to 49, wherein the probiotic is administered orally, by suppositories or through the gastric route by intubation.

51. A method according to claim 50 wherein the probiotic is in the form of a tablet, capsule, powder or liquid.

52. A method according to claim 51 wherein the tablet or capsule contains 10⁸ to 10¹² cfu of probiotic bacteria.

53. A method according to claim 50 wherein the probiotic is in the form of a food composition.

54. A method according to claim 53 wherein the food is a dairy or soy product.

55. A method according to claim 53 or claim 54, wherein the food composition contains 10⁸ to 10¹² cfu of probiotic bacteria per serve.

56. A method according to any one of claims 35 to 37, wherein the probiotic is administered in an amount of 5×10¹⁰ cfu every second day.

57. A method according to claim 56 wherein the probiotic is present in phosphate buffered saline (PBS).

58. A method according to claim 57 wherein the probiotic is administered by intragastric feed.

59. A method according to claim 35, wherein the effect of the probiotic on endotoxemia is determined by measurement of a parameter that is a direct and/or indirect indicator of the level of circulating endotoxin.

60. A method according to claim 36 wherein the effect of the probiotic on the liver condition is determined by measurement of a parameter that is a direct and/or indirect indicator of the level of circulating endotoxin.

61. A method according to claim 60 wherein the effect of the probiotic on the liver condition is determined by direct measurement of endotoxin levels.

62. A method according to 36 wherein alcohol is administered into the rectum of the mammal.

63. A method of controlling circulating endotoxin levels in a mammal including inhibiting growth and/or translocation of enteric Gram-negative bacteria by administering a probiotic, wherein the probiotic is probiotic bacteria selected from Lactobacillus species, with the proviso that the Lactobacillus species is not Lactobacillus GG.

64. A method according to claim 63 wherein the Lactobacillus is L. acidophilus.

65. A method according to claim 64, wherein L. acidophilus is the strain VRI-001.

66. A method according to claim 63 wherein the Lactobacillus is is L. fermentum.

67. A method according to claim 66 wherein the L. fermentum is strain VRI-002.

68. A method according to any one of claims 35 to 67 wherein the mammal is a human, horse, pig, a member of the ovine, bovine or poultry species or a companion animal such as a dog or a cat.

69. A method according to claim 1 or claim 35 wherein the endotoxemia results from translocation of Gram negative bacteria from the gut into other organs.

70. Use according to claim 20 wherein the endotoxemia results from translocation of Gram negative bacteria from the gut to other organs.

71. A method according to claim 60 wherein the circulating endotoxin is due to the translocation of Gram negative bacteria from the gut to other organs.
